(19) United States
(12) Patent Application Publication
(10) Pub. No.: US 2009/0156611 A1
(43) Pub. Date: Jun. 18, 2009

(54) MAMMALIAN HEDGEHOG SIGNALING MODULATORS

(75) Inventors: Antti Oinas, Helsinki (FI); Jussi Taipale, Espoo (FI); Juhani Lahdenperä, Helsinki (FI)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: Licentia Ltd., Helsinki (FI)

(21) Appl. No.: 12/093,182

(22) PCT Filed: Nov. 10, 2006
(86) PCT No.: PCT/FI06/50491

§ 371 Date: Sep. 9, 2008

Related U.S. Application Data
(60) Provisional application No. 60/735,288, filed on Nov. 11, 2005.

(51) Int. Cl. A61K 31/496; A61K 31/381; A61P 35/00; A61K 31/4965; A61K 31/17; A61K 31/4184; A61K 31/18; A61K 31/421; A61K 31/438; A61K 31/426; A61K 31/4439

(52) U.S. Cl. 514/254.1; 514/445; 514/255.01; 514/587; 514/394; 514/604; 514/374; 514/278; 514/370; 514/342

(57) ABSTRACT

The disclosure relates to compositions for and methods of inhibiting the mammalian Hedgehog signaling pathway.

### FIELD OF THE RELATED TECHNOLOGY

The present invention concerns small-molecule compounds having therapeutic utility as inhibitor of mammalian Hedgehog signaling, as well as compositions comprising the compounds, and methods of making and using the molecules and compositions.

### DESCRIPTION OF RELATED ART

The Hedgehog (Hh) is a family of secreted proteins that plays a central role in regulating cell differentiation, proliferation, and tissue patterning during development (Ingham & McMahon 2001). Hh was first identified in Drosophila flies, where it specifies a positional identity in embryonic segmentation. In mammals there are three Hh homologs, Sonic Hedgehog (Shh), Indian Hedgehog (Ihh) and Desert Hedgehog (Dhh). Of these three, Shh has been the focus of many studies because it has the broadest range of expression during the development, and because results from experiments with Shh apply also to Ihh and Dhh. Because of the central role that Shh plays in regulating cell differentiation and proliferation, the correct regulation of the pathway activity is critical both during development and in adults. The notion that Shh pathway misregulation is a reoccurring theme in different types of cancers in various tissues underlines this importance (Taipale & Beachy 2001).

Shh protein is secreted from cells expressing Shh. Before secretion, Shh protein undergoes an intramolecular cleavage and lipid modification catalyzed by the carboxy-terminal portion of the precursor. The result from the cleavage is an amino-terminal peptide with a mass of 19 kDa with a carboxy-terminal cholesterol molecule (ShhNp). After the cholesterol addition, ShhNp undergoes palmitoylation. The secreted ShhNp acts in para- and/or autocrine fashion on cells expressing the Shh receptor, Ptch. Ptch is a twelve-span transmembrane protein structurally similar to the putative proton-driven lipid translocator mutated in Niemann-Pick C1 disease. The Shh pathway differs from most signal pathways in that the signal transduction progresses in sequential repressive interactions. When extracellular Shh is not present, Ptch represses the activity of another transmembrane protein, Smo. Smo is a member of the seven-transmembrane receptor family, most closely related to the Fzd family of Wnt receptors. When Shh is bound to Ptch, it releases the suppression of Smo. Smo activation results in activation of transcriptional response mediated by the transcription factors of the GLI family. The intracellular pathway leading to activation of GLI family in mammals appears to differ from the corresponding pathway in flies. In flies, the key negative regulator acting downstream of Smo is kinesin-like protein Costal-2 (Cos2). Cos2 is a cytoplasmic protein associated with microtubules. It anchors the Hh regulatory complex which contains the serine/threonine protein kinase Fused (Fu), Suppressor of Fused (Su(fu)), and the drosophila homolog of GLI family Cubitus interruptus (Ci). In the absence of Hh, Ci is phosphorylated by protein kinase A (Pka) and subsequentially cleaved to generate an N-terminal transcriptional repressor. Stimulation with Hh leads to dissociation of the regulatory complex from the microtubules and to translocation of the full-length Ci to the nucleus, where it acts as a transcriptional activator of the Hh target genes. The mammalian homolog of Cos2 is not identified, and it appears that the mammalian homolog of Su(fu) acts as the key negative regulator of the pathway. In mammals the transcriptional activators appear to be Gli1 and Gli2 and the repressor function is executed by Gli3. All GLI transcription factors (GLIs 1, 2, and 3; this group of factors is referred to herein as GLI) mediate their effects through binding to sequences in regulatory elements of the Shh target genes. For the purposes herein, the mammalian Shh pathway is defined as components of the cellular signaling pathway acting on and including the Gli family of transcription factors. An inhibitor of this pathway is defined as a compound leading to lower levels of Gli activity than that observed in control cells with no compound present. An activator of this pathway is defined as a compound leading to higher levels of Gli activity compared to control with no compound present.

Mutations of the components of the Shh pathway in humans lead to severe diseases that result from either loss of function or ectopic activation of the pathway. Haploinsufficiency of human SHH or mutation in human PTCHI gene are associated with holoprosencephaly, a syndrome affecting development of the forebrain and mid-face (Roessler et al. 1996 & Ming et al. 2002). Ectopic expression of Shh, Gli1 or Gli2 in model systems leads to the formation of tumors that resemble basal cell carcinomas (BCC) (Ruiz i Altaba et al. 2002). Consistent with this it is known that sporadic human BCCs consistently express GLI, suggesting that all sporadic BCCs have this pathway active. Another kind of tumors is also associated with ectopic activation of the Shh pathway. Human mutations in the SU(FU) gene predispose the carrier with cerebellar cancer medulloblastoma (Taylor et al. 2002). Sporadic medulloblastomas carry often PTCH1 mutations and express GLI and mice heterozygous of Ptch null allele often develop medulloblastomas (Goodrich et al. 1997, Raffel et al. 1997, Pomeroy et al. 2002 & Ruiz i Altaba et at 2002). This observation is supported by the fact that Shh acts as a stem cell factor in the cerebellar external germinal layer (EGL) cells from which the medulloblastoma is thought to originate (Wechsler-Reya et al. 2001). Recent studies indicate that the activation of Shh pathway is observed in the stomach and other gastrointestinal cancers (Berman et al. 2003). Shh target genes have been reported to be activated in 63 of 99 primary gastric cancers (Ma et al. 2005). Also activation of target genes is reported in ovarian fibromas and ovarian dermoids (Levanat et al. 2004), in oral squamous cell carcinoma (OSCC) (Nishimaki et al. 2004), in small-cell lung cancer (SCLC) (Watkins et al. 2003). Also reported is the involvement of Shh signaling with prostate cancer (Sanchez et al. 2004), and rhabdomyosarcomas (Hahn et al. 2000). Shh signaling has recently been shown to be linked also with psoriasis (Meth et al. 2006).

Loss of Shh pathway activity is also implicated in human diseases. Supporting the possibility of treating neurodegenerative diseases like Parkinson's disease by activating the Shh pathway are findings in which Shh can induce dopaminergic neuronal differentiation (Wang et al. 1995 & Flynes ci al. 1995) and that an injection of Shh into the striatum of the rat model of the Parkinson's disease reduces the behavioral defects (Tsuboi & Shults 2002).

It has been previously shown that mutations leading to aberrant activation or inactivation of the Shh pathway can target different levels of the pathway. A plant-derived steroidal alkaloid, cyclopamine, antagonizes Shh signaling (Cooperci al. 1998) by binding directly to the Smo heptahelical domain (Chen et al. 2002). Binding of cyclopamine to Smo suppresses its activity and renders the cells insensitive to Shh. This leads to inactivation of the pathway and suppression of Shh target genes through Gli family of transcription factors.

Hh inhibitors have been reported to be inhibitors of angiogenesis (Surace et al. 2006) indicating that they may have a broad-spectrum anticancer effect.

A number of reports exist of purported Shh pathway inhibitors and uses thereof. See, e.g., U.S. Pat. Nos. 6,867,216; 6,686,388; 6,432,970; and 6,291,516; and U.S. Patent Publication Nos. 20040127474; 20040072914; 20040072913; and 20040110663; all of which are incorporated herein by reference for their teachings relating to activity assays and diagnostic, medical, and other uses for pathway inhibitor compounds.

Because of the clear importance of the Shh pathway, a need exists for additional inhibitors of the Shh pathway.

### SUMMARY OF THE INVENTION

The present invention addresses the aforementioned and other needs. For example, the invention provides novel small molecules and compositions as well as therapeutic compositions and uses of specific small-molecule compounds. Numerous molecules are described herein.

Compounds suitable for use with the disclosed methods and compositions include those having a formula as described below, specifically formulae (I)-(XI).

In one variation, the molecules themselves are the invention, preferably in a purified and/or isolated form. In another variation, the invention is a composition comprising one or more molecules of the invention—preferably purified and/or isolated—in admixture with a pharmaceutically acceptable diluent, adjuvant, excipient, or carrier. In another variation, the invention is a unit dosage formulation comprising a therapeutically effective amount of a molecule of the invention. In yet another variation, the invention is a sustained release formulation comprising a purified molecule of the invention.

Throughout this document, references to compounds of the invention should be understood to refer to the compounds themselves, and also pharmaceutically acceptable salts, esters, pro-drugs, and other formulations suitable for in vivo delivery of the active moiety to target cells.

Another aspect of the invention is a composition comprising two or more isolated compounds of the invention in admixture with each other. In preferred variations, the composition further comprises a diluent, adjuvant, excipient, or carrier.

The present invention also includes novel methods of treating patients suffering from cell proliferative disorders. An exemplary method of treatment comprises selecting a patient in need of treatment for a particular proliferative disorder, and administering to the patient an amount of a compound or composition of the invention effective to treat the disorder. The selecting the patient involves identifying the proliferative disorder by a review of a patient's medical records, a physical examination, a diagnostic test or interpretation of such test performed on the patient or on a biological sample (tissue, fluid, etc.) from the patient, or the like. The administering of the compound can be by any route of administration, many of which are described herein. Exemplary proliferative disorders, each of which is contemplated as a specific aspect of the invention, are described throughout the background, detailed description, and examples, including various patent and publication documents incorporated herein by reference. Proliferative disorders include, but are not limited to, malignant gliomas, breast cancer, basal cell carcinoma, medulloblastomas, neuroectodermal tumors, and ependymomas. The hedgehog antagonists can be used to cause transformed cells to become either post-mitotic or apoptotic.

In another variation, the selecting comprises both identifying the presence of the proliferative disorder and also screening the patient or a biological sample from the patient (e.g., a biopsy) for evidence of aberrant Shh pathway activity, where the selecting comprises choosing a patient with the disorder and with the aberrant activity.

In jurisdictions that forbid the patenting of methods that are practiced on the human body, the following restrictions are intended: (1) the selecting of a human subject shall be construed to be restricted to selecting based on testing of a biological sample that has previously been removed from a human body and/or based on information obtained from a medical history, patient interview, or other activity that is not practiced on the human body; and (2) the administering of a composition to a human subject shall be restricted to prescribing a controlled substance that a human subject will self-administer by any technique (e.g., orally, inhalation, topical application, injection, insertion, etc.); or that a person other than the prescribing authority shall administer to the subject. For each jurisdiction, the broadest reasonable interpretation that is consistent with laws or regulations defining patentable subject matter is intended. In jurisdictions that do not forbid the patenting of methods that are practiced on the human body, the selecting of subjects and the administering of compositions includes both methods practiced on the human body and also the foregoing activities.

Efficacy of treatment is indicated by one or more of the following, for a proliferative disorder: the slowing of cell proliferation, arresting cell proliferation, causing a reduction in proliferated cell mass, eliminating the proliferating cells, reducing or eliminating symptoms associated with cell proliferation, extending life and/or improving the quality of life.

As described herein in greater detail, modulation of the Shh pathway also may be efficacious for treatments where regeneration is desired, such as Parkinson's disease, Alzheimer's disease, nerve and spinal injuries, and bone repair. Another aspect of the invention is a method of treating patients suffering from any such disorder, e.g., a method comprising selecting a patient in need of treatment for the particular disorder, and administering to the patient an amount of a compound or composition of the invention effective to treat the disorder. In a preferred embodiment relating to the degenerative disorders, the selecting includes both identifying the disorder and confirming the presence of aberrant Shh activity.

For all methods and uses of the invention, co-therapy with two or more compounds of the invention, simultaneously or in tandem, also is contemplated.

In a related embodiment, the invention includes novel compositions for treatment of cell proliferative disorders and for inhibiting altered growth states of cells having specific loss-of- or gain-of-function phenotypes. Likewise, the invention includes use of molecules and compositions of the invention for the manufacture of a medicament for treatment of disorders described herein.

Additional features and variations of the invention will be apparent to those skilled in the art from the entirety of this application, including the drawing and detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

In addition to the foregoing, the invention includes, as an additional aspect, all embodiments of the invention narrower in scope in any way than the variations specifically mentioned above. For example, to the extent aspects of the invention have been described using ranges or genera for the sake of brevity, it should be understood that every sub-range, every individual value within a range, every subgenus, and every species are individually contemplated as a separate aspect of the invention. Likewise, various aspects and features of the invention can be combined, creating additional aspects which are intended to be within the scope of the invention. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIGS. 1 to 3 depict the structural formulas of twelve compounds (Hh-Antag 1 to Hh-Antag 12) of the invention, as well as the dose dependency curves of the compounds on a logarithmic scale, showing their respective ability to act as antagonists of the Shh pathway in the cell culture experiments with Shh-LIGHT2 cells.

### DETAILED DESCRIPTION OF THE INVENTION

In connection with the present invention, we have found that small molecules can act as inhibitors of the Shh pathway. The present compounds can be used to treat diseases resulting from aberrant activation or inactivation of the Shh pathway.

The present aromatic compounds are all formed by simple organic building elements, in preferred embodiments including at least one aromatic ring-structure, optionally containing a heteroatom, which are linked to other linear or cyclic elements of the compounds typically by single bonds, and they can easily and at a reasonable cost be synthesized. The molecular weight of the compounds is generally less than about 1500 Da, in particular less than about 1000 Da or even less than 500 Da. The compounds can be prepared by conventional methods of synthetic organic chemistry. Therefore the present invention provides a cost-efficient approach to providing novel therapeutically useful compounds.

As stated above, the present aromatic compounds are all formed by simple organic building elements, including at least one, most often two or more aromatic ring-structures, at least one of which may optionally contain a heteroatom selected from oxygen, nitrogen and sulfur. Typically, the aromatic rings have 5 to 7 members. The ring structures may also comprise two or more fused ring structures having 9 to 14 ring members. The aromatic rings are interlinked or they are linked to linear elements or to alicyclic elements of the compounds typically by single bonds which allows for flexibility of the molecule. Altogether, the molecule comprises 3 to 10 building elements, including aromatic rings and linear or alicyclic segments.

The present compounds are characterized as “small-molecular” compounds which means that they have a molecular weight of typically less than about 1500 Da, in particular less than about 1000 Da and preferably less than about 500 Da. They can be synthesized by conventional chemical reactions, as will be discussed in more detail below.

In the below formulas, “alkyl” refers to a linear or branched saturated hydrocarbon group containing 1 to 10 carbon atoms, for example methyl, ethyl, n-propyl, isopropyl, n-butyl, isobutyl, t-butyl, octyl, amyl, and the like. “Alkoxy” represents linear and branched saturated hydrocarbon group having 1 to 10 carbon atoms. “Lower alkoxy” designates alkoxy groups with 1 to 4 carbon atoms. Nonlimitng examples of alkoxy groups include methoxy, ethoxy, propoxy, and t-butoxy.

The term halogen, halo, and halide are used in the conventional sense to refer to a chloro, bromo, fluoro, or iodo substituent or corresponding ion.

“Ar” refers to an aryl group which represents an aromatic moiety generally containing 6 to 30 carbon atoms or can refer to a heteroaryl group. An aryl group can contain a single aromatic ring or multiple aromatic rings that are fused together, directly linked, or indirectly linked (such that the different aromatic rings are bound to a common group such as a methylene or ethylene moiety). Preferred aryl groups contain 6 to 20 carbon atoms, and particularly preferred aryl groups contain 6 to 12 carbon atoms. Nonlimiting examples of aryl groups containing one aromatic ring or two or more fused or linked aromatic rings include phenyl, naphthyl, biphenyl, diphenyl ether, diphenylamine, benzophenone, and the like. Aryl groups can optionally be substituted with one or more substituent groups. Nonlimiting examples of subsituent groups include halo, nitro, cyano, linear or branched alkyl, linear or branched alkenyl, aryl, cycloalkyl, cycloalkenyl, amino, amido, carboxylate, and hydroxy.

As used herein, heteroaryl is an aromatic moiety as defined above for aryl, and that further contains at least one ring heteroatom selected from the group consisting of oxygen, nitrogen, and sulfur. Non-limiting examples of heteroaryl groups include pyrrolyl, pyrrolidinyl, pyridinyl, quinolinyl, indolyl, pyrimidinyl, furyl, thiophenyl, oxazolyl, azolyl, imidazolyl, 1,2,4-triazolyl, tetrazolyl, 2,2′-bipyridinyl, and pyridine[3,2,h]quinolinyl.

According to one embodiment, the present invention provides the following compounds and methods of modulating mammalian Hedgehog signaling by administering compounds having the structure (I′) and pharmaceutically acceptable salts thereof, a specific example of a compound of formula (I′) is formula (I):

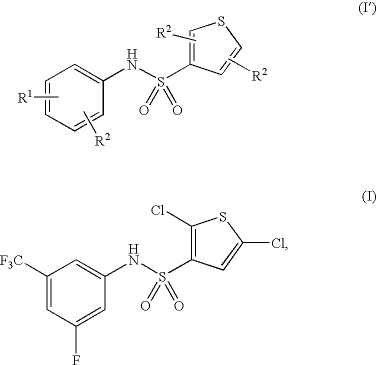

wherein R¹ is hydrogen or an unsubstituted or substituted, linear or branched C₁₋₁₀ alkyl; and R² is selected from the group of hydrogen, halogen and unsubstituted or substituted, linear or branched C₁₋₁₀ alkyl and C₁₋₁₀ alkoxy groups.

Additional compounds have the structure (II′), or more specifically (II):

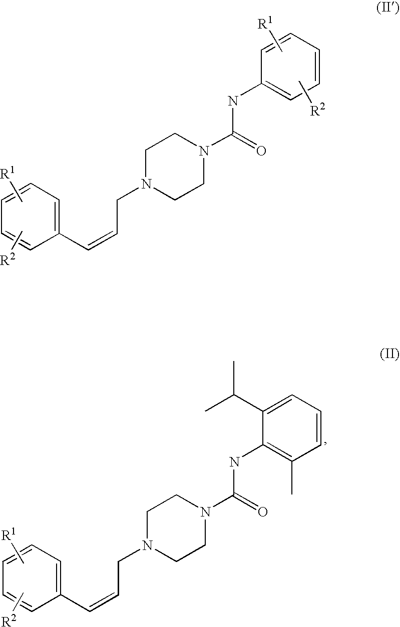

wherein R¹ is hydrogen or an unsubstituted or substituted, linear or branched C₁₋₁₀ alkyl, and
R² is selected from the group of hydrogen, halogen and unsubstituted or substituted, linear or branched C₁₋₁₀ alkyl and C₁₋₁₀ alkoxy groups. Nonlimiting examples of R¹ substituents include hydrogen, ethyl, n-propyl and n-amyl. Nonlimiting examples of R² substituents include hydrogen, chlorine, methyl and methoxy. In some embodiments, both R¹ and R² are hydrogen.

Pharmaceutically acceptable salts or esters also are contemplated for any and/or all compound disclosed herein.

According to another embodiment, the invention provides compounds having the structure (III′), or more specifically structure (III):

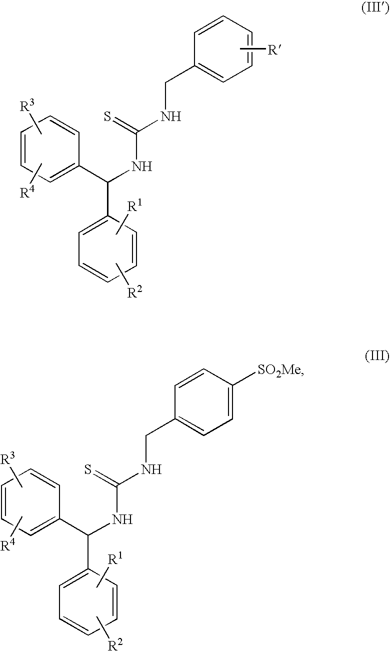

wherein R¹ is selected from the group of hydrogen and an unsubstituted or substituted, linear or branched C₁₋₁₀ alkyl groups; R² is selected from the group of hydrogen, halogen and unsubstituted or substituted, linear or branched C₁₋₁₀ alkyl and C₁₋₁₀ alkoxy groups; R³ is selected from the group of hydrogen, halogen, unsubstituted or substituted, linear or branched C₁₋₁₀ alkyl groups; R⁴ is selected from the group of hydrogen, halogen and unsubstituted or substituted, linear or branched C₁₋₁₀ alkyl and C₁₋₁₀ alkoxy groups; and R′ is hydrogen, halogen, unsubstituted or substituted, linear or branched C₁₋₁₀ alkyl and C₁₋₁₀ alkoxy groups, or SO₂R¹.

Additional compounds of the invention comprises the general formula (IV):

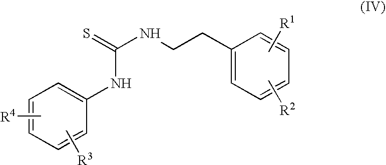

wherein R¹, R², R³, and R⁴ are as defined above. One compound of formula IV may include R¹ and/or R³ as a halogen and R² and/or R⁴ as an alkoxy group.

One example is the compound of formula IVa, below:

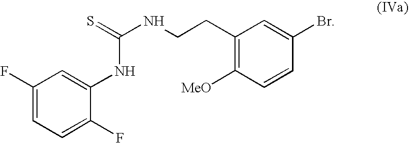

Further examples of compounds useful as inhibitors of mammalian Hedgehog signaling are the following:

a compound having the structure (V′), or more specifically (V):

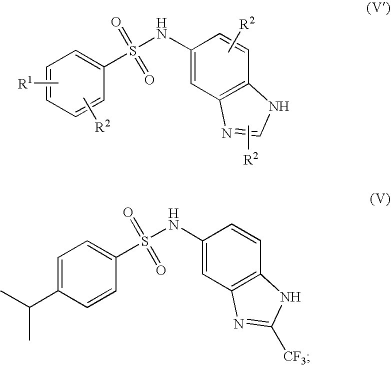

where R¹ and R² are as defined above;
a compound having the structure (VI′), or more specifically (VI):

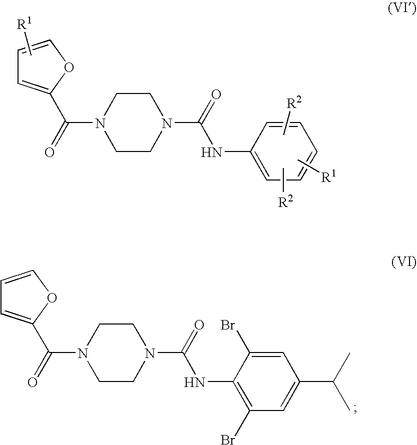

where R¹ and R² are as defined above;
a compound having the structure (VII′), or more specifically (VII):

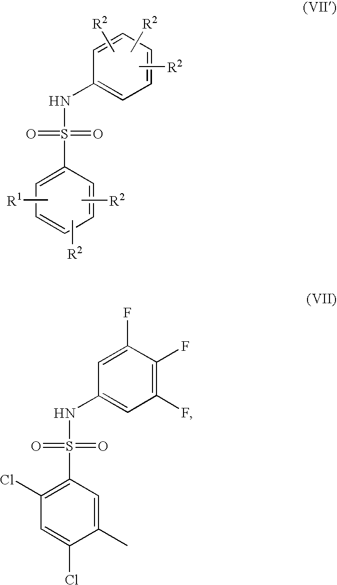

where R¹ and R² are as defined above;
a compound having the structure (VIII′), or more specifically (VIII):

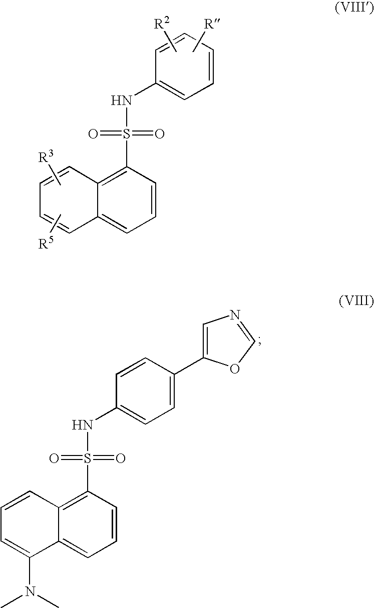

where R² and R³ are as defined above, R″ is hydrogen, halogen, unsubstituted or substituted linear or branched C₁₋₁₀ alkyl groups, or an unsubstituted or substituted aryl or heteroaryl group, and R⁵ is hydrogen, halogen, unsubstituted or substituted linear or branched C₁₋₁₀ alkyl groups, or N(R¹)₂;
a compound having the structure (IX′), or more specifically (IX):

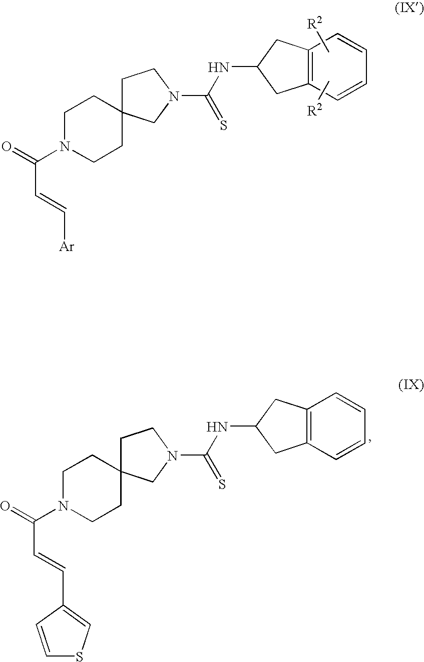

where R² is as defined above and Ar is an aryl or heteroaryl group optionally substituted; and a compound having the structure (X′), or more specifically (X):

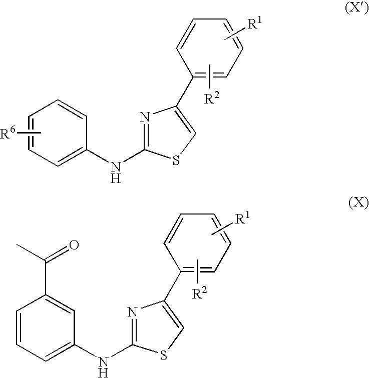

wherein R¹ and R² are as defined above, and R⁶ is hydrogen, halogen, unsubstituted or substituted, linear or branched C₁₋₁₀ alkyl groups, N(R¹)₂, or C(O)R¹. One compound of formula (X) may include R¹ and R² as hydrogen.

The above compounds of formulae (I)-(X) also may be as a pharmaceutically acceptable salt or ester.

A group of compounds useful as inhibitors of the mammalian Hedgehog pathway are represented by compounds having the structure (XI):

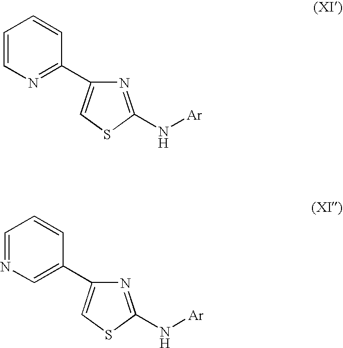

where Ar is an aryl or heteroaryl group, optionally substituted with one or more R² groups. Compounds of formulae (XI′) and (XI″) may be as pharmaceutically acceptable salts or esters thereof for use as inhibitors.

In formula XI, Ar is an aryl group and may contain a single ring or up to three fused aromatic rings, with one, two, or three substituents. These substituents may include halogen and unsubstituted or substituted, linear or branched C₁₋₁₀ alkyl and C₁₋₁₀ alkoxy groups. In some cases, Ar stands for a phenyl group substituted with 1 to 3 substituents such as halogen, C₁₋₄ alkyl, and C₁₋₄ alkoxy. Specific examples of compound of formula XI are as follows:

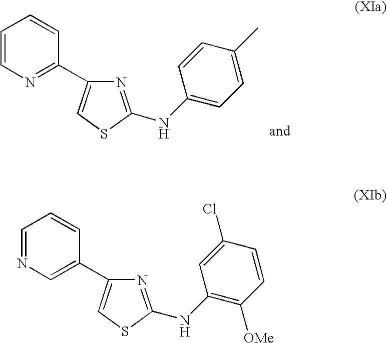

The present compounds and derivatives thereof are readily prepared by conventional synthetic methods. Conventional methods employed in combinatorial chemistry can be used for carrying out coupling reactions of the organic building blocks. In general, combinatorial chemistry techniques are laid out in a variety of publications, including U.S. Pat. Nos. 5,359,115, 5,362,899, 5,573,905, 5,712,171, and 5,736,412; WO 93/09668, WO 92/10092, and WO 91/07087, each of which is incorporated by reference in its entirety. Commercially available or easily synthesized starting materials, or building blocks, are used in the combinatorial chemistry techniques to obtain the compounds disclosed herein.

The compounds of formulae (I)-(XI) may be modulators of the Hedgehog signaling pathway, either as antagonists or agonists.

The modulation of the Hedgehog signaling pathway may be measured by determining the IC₅₀ of a particular compound of formulae (I)-(XI). The term “IC₅₀” is defined as the concentration of a compound that results in 50% enzyme inhibition, in a single dose response experiment. In the present invention, the IC₅₀ value is a measure of the potency of a compound to inhibit a Hedgehog protein, including Shh, Ihh, and Dhh. Determining the IC₅₀ value of a compound is readily carried out by a known in vitro methodology generally described in Cheng et al., Biochem. Pharmacology, 22, pages 3099-3108 (1973).

The compounds of formulae (I)-(XI) may be used in the treatment of disorders relating to cell proliferation, such as cancer and tumor therapy or diagnostics. The compounds of formulae (I)-(XI) may be used in diagnosing, treating, or ameliorating various cancers or other cell-proliferation disorders, such as basal cell carcinomas, medulloblastoma, gastrointestinal cancers, ovarian fibromas and ovarian dermoids, oral squamous cell carcinoma (OSCC), small-cell lung cancer (SCLC), prostate cancer, and rhabdomyosarcomas. Additional disorders are identified in the examples, below.

Disclosed herein are compositions of the compounds as described above. The compositions comprise a therapeutically effective amount of the compounds or pharmaceutically acceptable salts thereof and a pharmaceutically acceptable carrier, adjuvant, and/or diluent.

The inhibitors are employed in amounts effective to achieve their intended purpose. As used herein, a “therapeutically effective amount” means an amount effective to inhibit development of, or to alleviate the existing symptoms of, the condition of the subject being treated. “Dose-effective to inhibit” means an amount effective to inhibit the Hedgehog signaling pathway, in vivo or ex vivo. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which is expressed as the ratio of LD₅₀ to ED₅₀. Compounds that exhibit high therapeutic indices (i.e., a toxic dose that is substantially higher than the effective dose) are preferred.

Inhibition of the Hedgehog signaling pathway can measured using a dose-response assay in which a sensitive assay system is contacted with a compound of interest over a range of concentrations, including concentrations at which no or minimal effect is observed, through higher concentrations at which partial effect is observed, to saturating concentrations at which a maximum effect is observed. Theoretically, such assays of the dose-response effect of inhibitor compounds can be described as a sigmoidal curve expressing a degree of inhibition as a function of concentration. The curve also theoretically passes through a point at which the concentration is sufficient to reduce activity of the Hedgehog signaling pathway to a level that is 50% that of the difference between minimal and maximal activity in the assay. This concentration is defined as the Inhibitory Concentration (50%) or IC₅₀ value. Determination of IC₅₀ values preferably is made using conventional biochemical (acellular) assay techniques or cell based assay techniques.

Comparisons of the efficacy of inhibitors often are provided with reference to comparative IC₅₀ values, wherein a higher IC₅₀ indicates that the test compound is less potent, and a lower IC₅₀ indicates that the compound is more potent, than a reference compound. Inhibitor compounds demonstrating IC₅₀ values of less than about 1000 nM, or less than about 250 nM, or less than about 100 nM, or less than about 50 nM, or less than about 20 nM, or less than about 1 nM, when measured using the dose-response assay, may be employed in compositions or methods according to the invention.

The data obtained in such dose-response assays can be used as a factor in formulating a dosage range for use in mammals, and more specifically, humans. The dosage of such compounds preferably lies within a range of circulating concentrations that include the ED₅₀ with little or no toxicity. The dosage can vary within this range depending upon the dosage form, and the route of administration utilized.

The exact formulation, route of administration, and dosage is chosen by a subject's physician, or treating professional, in view of the subject's condition. Dosage amount and interval can be adjusted individually to provide plasma levels of the active compound that are sufficient to maintain desired therapeutic effects. In general, however, doses employed for humans typically are in the range of 0.001 mg/kg to about 1000 mg/kg per day, in a range of about 0.1 mg/kg to about 500 mg/kg per dose of inhibitor. In some embodiments, doses range from about 0.1 to about 50 mg/kg, about 0.5 to about 40 mg/kg, about 0.7 to about 30 mg/kg, or about 1 to about 20 mg/kg. Specific doses contemplated include sub-ranges of any of the foregoing ranges in 0.1 mg/kg increments.

As used herein, the term “pharmaceutically acceptable salts” refers to salts or zwitterionic forms of the compounds a described above. Salts of such compounds can be prepared during the final isolation and purification of the compounds or separately by reacting the compound with an acid having a suitable cation. Suitable pharmaceutically acceptable cations include alkali metal (e.g., sodium or potassium) and alkaline earth metal (e.g., calcium or magnesium) cations. In addition, the pharmaceutically acceptable salts of the disclosed compounds that contain a basic center are acid addition salts formed with pharmaceutically acceptable acids. Examples of acids which can be employed to form pharmaceutically acceptable salts include inorganic acids such as hydrochloric, hydrobromic, sulfuric, and phosphoric, and organic acids such as oxalic, maleic, succinic, malonic, and citric. Nonlimiting examples of salts of compounds of the invention include, but are not limited to, hydrochloride, hydrobromide, hydroiodide, sulfate, bisulfate, 2-hydroxyethansulfonate, phosphate, hydrogen phosphate, acetate, adipate, alginate, aspartate, benzoate, butyrate, camphorate, camphorsulfonate, citrate, digluconate, glycerolphosphate, hemisulfate, heptanoate, hexanoate, formate, succinate, malonate, fumarate, maleate, methanesulfonate, mesitylenesulfonate, naphthylenesulfonate, nicotinate, oxalate, pamoate, pectinate, persulfate, 3-phenylpropionate, picrate, pivalate, propionate, trichloroacetate, trifluoroacetate, glutamate, bicarbonate, undecanoate, lactate, citrate, tartrate, gluconate, benzene sulphonate, and p-toluenesulphonate salts. In addition, available amino groups present in the compounds of the invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference to compounds appearing herein is intended to include compounds disclosed herein as well as pharmaceutically acceptable salts, solvates (e.g., hydrates), esters, or prodrugs thereof.

The pharmaceutical compositions may be in the form of a aqueous, oleaginous suspension, dispersions or sterile powders, which may be used for the extemporaneous preparation of injectable solutions or dispersions. The suspension may be formulated according to the known art using those suitable dispersing or wetting agents and suspending agents which have been mentioned above. The compositions may also be solution or suspension in a non-toxic diluent or solvent, for example as a solution in 1,3-butane diol. The carrier can be a solvent or dispersion medium containing, for example, water, ethanol, polyol (for example, glycerol, propylene glycol, and liquid polyethylene glycol, and the like), suitable mixtures thereof, vegetable oils, Ringer's solution and isotonic sodium chloride solution. In addition, fixed oils may be employed as a solvent or suspending medium. For this purpose any bland fixed oil may be employed including synthetic mono- or diglycerides. In addition, fatty acids such as oleic acid find use in the preparation of injectables.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose, or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides (preferably sodium or potassium chloride); delivery vehicles; diluents; excipients and/or pharmaceutical adjuvants. (Remington's Pharmaceutical Sciences, 18th Edition, A. R. Gennaro, ed., Mack Publishing Company (1990).

The optimal pharmaceutical composition will be determined by one skilled in the art depending upon, for example, the intended route of administration, delivery format, and desired dosage. See, for example, Remington's Pharmaceutical Sciences, supra. Such compositions may influence the physical state, stability, rate of in vivo release, and rate of in vivo clearance of the mammalian hedgehog inhibitor.

The primary vehicle or carrier in a pharmaceutical composition may be either aqueous or non-aqueous in nature. For example, a suitable vehicle or carrier may be water for injection, physiological saline solution, solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefore.

The pharmaceutical compositions can be selected for parenteral delivery. Alternatively, the compositions may be selected for inhalation or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use in this invention may be in the form of a pyrogen-free, parenterally acceptable aqueous solution comprising the Hedgehog inhibitor in a pharmaceutically acceptable vehicle. A particularly suitable vehicle for parenteral injection is sterile distilled water in which a Hedgehog inhibitor is formulated as a sterile, isotonic solution, properly preserved. Yet another preparation can involve the formulation of the desired molecule with an agent, such as injectable microspheres, bio-erodible particles, polymeric compounds (such as polylactic or polyglycolic acid), or beads or liposomes, that provide for the controlled or sustained release of the product which may then be delivered via a depot injection. Hyaluronic acid may also be used, and this may have the effect of promoting sustained duration in the circulation. Other suitable means for the introduction of the desired molecule include implantable drug delivery devices.

In one embodiment, a pharmaceutical composition may be formulated for inhalation. For example, a Hedgehog inhibitor may be formulated as a dry powder for inhalation. Inhalation solutions may also be formulated with a propellant for aerosol delivery. In yet another embodiment, solutions may be nebulized. Pulmonary administration is further described in PCT application no. PCT/US94/001875, which describes pulmonary delivery of chemically modified proteins, but which may be applicable to pulmonary delivery of compounds as disclosed herein.

It is also contemplated that certain formulations may be administered orally. In one embodiment of the present invention, Hedgehog inhibitors which are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the Hedgehog inhibitor. Diluents, flavorings, low melting point waxes, vegetable oils, lubricants, suspending agents, tablet disintegrating agents, and binders may also be employed.

Another pharmaceutical composition may involve an effective quantity of Hedgehog inhibitor in a mixture with non-toxic excipients which are suitable for the manufacture of tablets. By dissolving the tablets in sterile water, or another appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving Hedgehog inhibitors in sustained- or controlled-delivery formulations. Techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. Additional examples of sustained-sustained-release preparations include semipermeable polymer matrices in the form of shaped articles, e.g. films, or microcapsules. Sustained release matrices may include polyesters, hydrogels, polylactides (U.S. Pat. No. 3,773,919 and EP 058 481), copolymers of glutamic acid and gamma ethyl-L-glutamate (Sidman et al., Biopolymers, 22:547-556 (1983)), poly(2-hydroxyethyl-methacrylate) (Langer et al., J. Biomed. Mater. Res., 15:167-277 (1981) and Langer, Chem. Tech., 12:98-105 (1982)), ethylene vinyl acetate (Langer et al., supra) or poly-D-3-hydroxybutyric acid (EP 133 988). Sustained-release compositions may also may include liposomes, which can be prepared by any of several methods known in the art. See e.g., Eppstein et al., Proc. Natl. Acad. Sci. USA, 82:3688-3692 (1985); EP 88 046; 036 676; and EP 143,949.

The pharmaceutical composition to be used for in vivo administration typically must be sterile. This may be accomplished by filtration through sterile filtration membranes. Where the composition is lyophilized, sterilization using these methods may be conducted either prior to, or following lyophilization and reconstitution. The composition for parenteral administration may be stored in lyophilized form or in a solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or as a dehydrated or lyophilized powder. Such formulations may be stored either in a ready-to-use form or in a form (e.g., lyophilized) requiring reconstitution prior to administration.

Further disclosed herein are methods of modulating mammalian hedgehog activity comprising administering to a subject in need thereof a inhibitor having a formula of one of formulae (I)-(XI), as described above. The subject in need thereof is typically a mammal, and in some specific embodiments, is a human.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

Further utility of the present compounds is in the field of biological research as chemicals, including reagents for testing of biological models.

Based on the above valuable properties, the present compounds can be used for manufacturing medicaments having biological and therapeutic activity, in particular for modulation of the mammalian Hedgehog signaling pathway.

The compounds employed in the methods of the present invention may be administered by any means that results in the contact of the active agent with the agent's site of action in the body of a patient. The compounds may be administered by any conventional means available for use in conjunction with pharmaceuticals, either as individual therapeutic agents or in a combination of therapeutic agents. For example, they may be administered as the sole active agent in a pharmaceutical composition, or they can be used in combination with other therapeutically active ingredients.

Compounds of the present invention can be administered to a mammalian host in a variety of forms adapted to the chosen route of administration, e.g., orally or parenterally. Parenteral administration in this respect includes administration by the following routes: intravenous, intramuscular, subcutaneous, rectal, intraocular, intrasynovial, transepithelial including transdermal, ophthalmic, sublingual and buccal; topically including ophthalmic, dermal, ocular, rectal, and nasal inhalation via insufflation aerosol. The active compound may be orally administered, for example, with an inert diluent or with an assimilable edible carrier or excipient, or it may be enclosed in hard or soft shell gelatin capsules, or it may be compressed into tablets, or it may be incorporated directly with the food of the diet. For oral therapeutic administration, the active compound may be incorporated with excipient and used in the form of ingestible tablets, buccal tablets, troches, capsules, elixirs, suspensions, syrups, wafers, and the like.

Pharmaceutically acceptable ingredients are well known for the various types of formulation and may be for example binders such as natural or synthetic polymers, excipients, lubricants, surfactants, sweetening and flavouring agents, coating materials, preservatives, dyes, thickeners, adjuvants, antimicrobial agents, antioxidants and carriers for the various formulation types. Nonlimiting examples of binders useful in a composition described herein include gum tragacanth, acacia, starch, gelatine, and biological degradable polymers such as homo- or co-polyesters of dicarboxylic acids, alkylene glycols, polyalkylene glycols and/or aliphatic hydroxylcarboxylic acids; homo- or co-polyamides of dicarboxylic acids, alkylene diamines, and/or aliphatic amino carboxylic acids; corresponding polyester-polyamide-co-polymers, polyanhydrides, polyorthoesters, polyphosphazene and polycarbonates. The biological degradable polymers may be linear, branched or crosslinked. Specific examples are poly-glycolic acid, poly-lactic acid, and poly-d,1-lactide/glycolide. Other examples for polymers are water-soluble polymers such as polyoxaalkylenes (polyoxaethylene, polyoxapropylene and mixed polymers thereof, poly-acrylamides and hydroxylalkylated polyacrylamides, poly-maleic acid and esters or -amides thereof, poly-acrylic acid and esters or -amides thereof, poly-vinylalcohol und esters or -ethers thereof, poly-vinylimidazole, poly-vinylpyrrolidon, und natural polymers like chitosan.

Nonlimiting examples of excipients useful in a composition described herein include phosphates such as dicalcium phosphate. Nonlimiting examples of lubricants use in a composition described herein include natural or synthetic oils, fats, waxes, or fatty acid salts such as magnesium stearate.

Surfactants for use in a composition described herein can be anionic, anionic, amphoteric or neutral. Nonlimiting examples of surfactants useful in a composition described herein include lecithin, phospholipids, octyl sulfate, decyl sulfate, dodecyl sulfate, tetradecyl sulfate, hexadecyl sulfate and octadecyl sulfate, Na oleate or Na caprate, 1-acylaminoethane-2-sulfonic acids, such as 1-octanoylaminoethane-2-sulfonic acid, 1-decanoylaminoethane-2-sulfonic acid, 1-dodecanoylaminoethane-2-sulfonic acid, 1-tetradecanoylaminoethane-2-sulfonic acid, 1-hexadecanoylaminoethane-2-sulfonic acid, and 1-octadecanoylaminoethane-2-sulfonic acid, and taurocholic acid and taurodeoxycholic acid, bile acids and their salts, such as cholic acid, deoxycholic acid and sodium glycocholates, sodium caprate or sodium laurate, sodium oleate, sodium lauryl sulphate, sodium cetyl sulphate, sulfated castor oil and sodium dioctylsulfosuccinate, cocamidopropylbetaine and laurylbetaine, fatty alcohols, cholesterols, glycerol mono- or -distearate, glycerol mono- or -dioleate and glycerol mono- or -dipalmitate, and polyoxyethylene stearate.

Nonlimiting examples of sweetening agents useful in a composition described herein include sucrose, fructose, lactose or aspartame. Nonlimiting examples of flavoring agents for use in a composition described herein include peppermint, oil of wintergreen or fruit flavors such as cherry or orange flavor. Nonlimiting examples of coating materials for use in a composition described herein include gelatin, wax, shellac, sugar or other biological degradable polymers. Nonlimiting examples of preservatives for use in a composition described herein include methyl or propylparabens, sorbic acid, chlorobutanol, phenol and thimerosal.

Such compositions and preparations should preferably contain at least about 0.1% by weight of active compound. The dosage of the compounds of the present invention that will be most suitable will vary with the form of administration, the particular compound chosen and the physiological characteristics of the particular patient under treatment. In some cases, the compositions or preparations contain a compound of formula (I)-(XI) in the range of about 2% to about 6%. The amount of active compound in the compositions or preparations may be selected so as to provide a suitable dosage for the disorder, disease or diagnostic application. Compositions or preparations according to the present invention may be prepared so that an oral dosage unit form contains from about 0.1 to about 1000 mg of active compound, and all combinations and subcombinations of ranges and specific amounts therein.

The active compounds of formula (I)-(XI) may be formulated into tablets, troches, pills, capsules and the like and may further a binder, such as gum tragacanth, acacia, corn starch or gelatin; an excipient, such as dicalcium phosphate; a disintegrating agent, such as corn starch, potato starch, alginic acid and the like; a lubricant, such as magnesium stearate; a sweetening agent such as sucrose, lactose or saccharin; or a flavoring agent, such as peppermint, oil of wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propyl parabens as preservatives, a dye and flavoring, such as cherry or orange flavor. The active compound may be incorporated into sustained-release preparations and formulations.

The active compound may be administered parenterally or intraperitoneally. Solutions of the active compound as a free base or a pharmacologically acceptable salt can be prepared in water suitably mixed with a surfactant, such as hydroxypropylcellulose. A dispersion can also be prepared in glycerol, liquid polyethylene glycols and mixtures thereof and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the active compound in the required amount, in the appropriate solvent, with various of the other ingredients enumerated above, as required, followed by filtered sterilization. Generally, dispersions may be prepared by incorporating the sterilized active ingredient into a sterile vehicle that contains the basic dispersion medium and the required other ingredients from those enumerated above. In the case of sterile powders for the preparation of sterile injectable solutions, the preferred methods of preparation may include vacuum drying and the freeze drying technique, which yield a powder of the active ingredient, plus any additional desired ingredient from the previously sterile-filtered solution thereof.

The therapeutic compounds of this invention may be administered to a patient alone or in combination with a pharmaceutically acceptable carrier. As noted above, the relative proportions of active ingredient and carrier may be determined, for example, by the solubility and chemical nature of the compound, chosen route of administration and standard pharmaceutical practice.

Furthermore, the compounds of this invention can, when used in cancer therapy, be used together with other substances and compounds, such as chemotherapeutic agents. Such compounds are, for example (according to the general classes of the compounds): Alkylating agents, such as cyclophosphamide, cisplatin, carboplatin, ifosfamide, chlorambucil, busulfan, thiotepa, nitrosoureas);

Anti-metabolites, such as 5-fluorouracil, fludarabine, methotrexate, azathioprine, gemcitabine (Gemzar);
Antitumour antibiotics, such as doxorubicin, daunorubicin, epirubicin, actinomycin, bleomycin, mitomycin, plicamycin, dactinomycin, adriamycin;
Hormonal therapy, such as steroids, finasteride, aromatase inhibitors, tamoxifen, goserelin;
Taxanes, such as paclitaxel (Taxol), docetaxel (Taxotere);
(antimicrotubule);
Topoisomerase inhibitors, such as irinotecan, topotecan, amsacrine, etoposide, etoposide phosphate, teniposide;
Vinca alkaloids, such as vincristine, vinblastine, vinorelbine, vindesine;
(antimiotic agents)
capecitabine (Xeloda), podophyllotoxin, amoxicillin, and piroxicam.

In addition to the above, there are several novel compounds disclosed in pending patent applications, e.g.: Epothilones (US 2005244413), serratamolide (US 2005239694), indol derivatives (US 2005239752), various plant extracts: extract of sea buckthorn—Hippophae rhamnoides (US 2005214394), extracts of Ganoderma lucidum, Salvia miltiorrhiza and Scutellaria barbata (US 2005208070), the contents of the afore-mentioned US Patent Applications are herewith incorporated by reference.

In particular, administration can be carried out parenterally, for example by i.v., i.c.v. (intracerebroventricularly) and i.m. administration. Parenteral compositions usually contain a buffering agent and, optionally, a stabilizing agent.

When necessary, in order to promote penetration of the blood-brain-barrier, the active compounds can be administered by using various now strategies for gaining drug access to the brain. These include the transcellular lipophilic pathway, which allows small, lipophilic compounds to cross the blood-brain barrier. A second pathway is “receptor-mediated endocytosis.” Further, as known in the art, some experimental work has shown that a monoclonal antibody for the transferrin receptor, coupled with brain-derived neurotrophin factor, which is neuroprotective but cannot cross the barrier itself, can both cross the barrier and exert neuroprotective effects. Endothelial cells of the blood-brain barrier also express a number of transport proteins, including transporters for glucose, amino acids, nucleosides, and other compounds. Thus, to focus on the latter strategy, the compounds can be designed such that they gain access to the brain by going through these transport processes. It is, however, also possible to block these processes, in that way bolstering brain levels of endogenous permeant.

The compounds employed in the uses and methods of the present invention may exist in prodrug form. As used herein, the term “prodrug” is intended to include any covalently bonded carriers which release the active parent drug or other formulas or compounds employed in the methods of the present invention in vivo when such prodrug is administered to a mammalian subject. Since prodrugs are known to enhance numerous desirable qualities of pharmaceuticals (e.g., solubility, bioavailability, manufacturing, etc.), the compounds employed in the present methods may, if desired, be delivered in prodrug form. Thus, the present invention contemplates methods of delivering prodrugs. Prodrugs of the compounds employed in the present invention may be prepared by modifying functional groups present in the compound in such a way that the modifications are cleaved, either in routine manipulation or in vivo, to the parent compound.

Accordingly, prodrugs include, for example, compounds described herein in which a hydroxy, thiol, amino, or carboxy group is bonded to any group that, when the prodrug is administered to a mammalian subject, cleaves to form a free hydroxyl, thiol, free amino, or carboxylic acid, respectively. Examples include, but are not limited to, acetoxyalkyls, acetate, formate and benzoate derivatives of alcohol, thiol, and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

### EXAMPLES

The experiments and experimental procedures set forth below provide guidance for analysis of the compounds of formulae (I)-(XI) as inhibitors of the Hedgehog signaling pathway.

### Materials and Methods

Chemical library: The chemicals used in the experiment were part of diversity-orientated library of small molecular weight compounds purchased from Tripos, Inc. (St. Louis, USA) and Chemical Diversity Labs, Inc. (San Diego, USA).

Cultured cell line assays: The cell line used to assay the effects of compounds on activity of mammalian Hedgehog pathway was Shh-LIGHT2 (AflC; Manassas, USA). The cell line was maintained in DMEM with 10% Normal Calf Serum (Hyclone; Logan, USA), L-Glutamine, Penicillin and Streptomycin. The Shh-LIGHT2 cell line is derived from mouse NIH/3T3 cell line. NIH/3T3 cells were co-transfected with GLI-responsive Firefly luciferase reporter (Sasaki et al. 1997) and pSV-Neo. A stable clonal cell line was selected using G418. The resulting cell line has been transfected with pRL-TK constituitive Renilla-luciferase expression vector (Promega; Madison, USA) and pVgRXR vector (Invitrogen; Carlsbad, USA) encoding the ecdysone receptor and a Zeocin recistance marker. Zeocin selection and cell cloning was then used to generate Shh-LIGHT2 cells. The cells were plated at 50% confluency on 96-well white culture plates in growth medium. After three days, for the screen for inhibitors the cells were changed to 0.5% serum-containing medium with recombinant ShhNp enough to fully activate the Hedgehog pathway. Pathway agonists were screened for in 0.5% serum containing medium without ShhNp. After medium exchange the compounds were transferred on cells at a concentration of 2-4 μM using a robotic pin tool system. After 2 days the medium containing the compounds was discarded, cells washed with 1× phosphate buffered saline, and lysed with Passive Lysis Buffer (Promega). Lysed cells were analysed for luminescence with Dual-Luciferase Reporter Assay System (Promega) using a Fluostar Optima microplate based multi-detection reader (BMG Labtech; Offenburg, Germany).

Recombinant Hedgehog protein: The ShhNp protein used in the study was amino-terminal human Shh produced in human HEK-293 cells. This Shh protein from Shh-N-producing HEK 293 cells is described in Chen et al. 2002. Shh polypeptide is described also in U.S. Pat. No. 6,132,728 and in U.S. Pat. No. 6,664,075, the contents of which are herein incorporated by reference.

### Example 1

### Isolation of Mammalian Hedgehog Pathway Antagonists by High-Throughtput Screening

Using the Shh-LIGHT2 cell line based assay 12 compounds were isolated from the 6000 compound library which can act as antagonists of the Shh pathway. As a criterion for antagonist we used a limit of half-maximal pathway inhibition (IC₅₀) of ≦1 μM. The IC₅₀ values of the isolated compounds ranged from 100 nM to 1 μM.

The GLI-dependent Firefly luciferase activity was used to measure the activity of the Shh pathway. Increased luciferase activity resulting from Gli dependend luciferase gene transcription was taken as a measure of Shh pathway activity.

Firefly luciferase activity was normalized with Renilla-luciferase activity to take account the difference in cell number and viability. Normalization with Renilla-luciferase activity was also used to dismiss false positive hits resulting from decreased firefly luciferase activity due to toxicity of assayed compound.

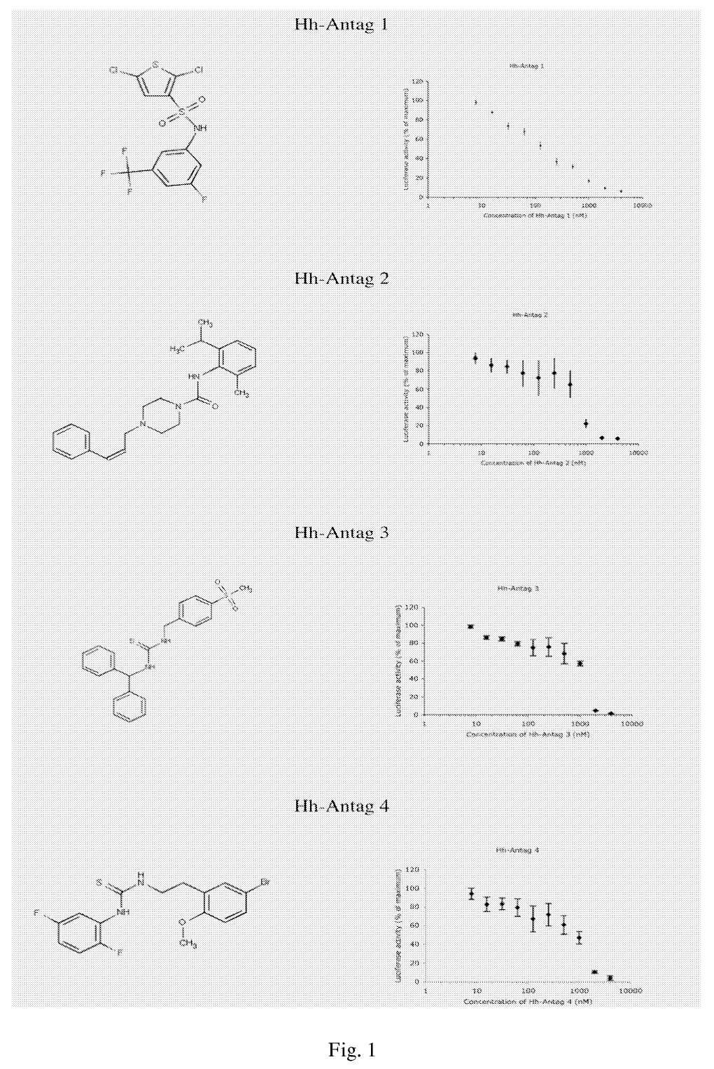

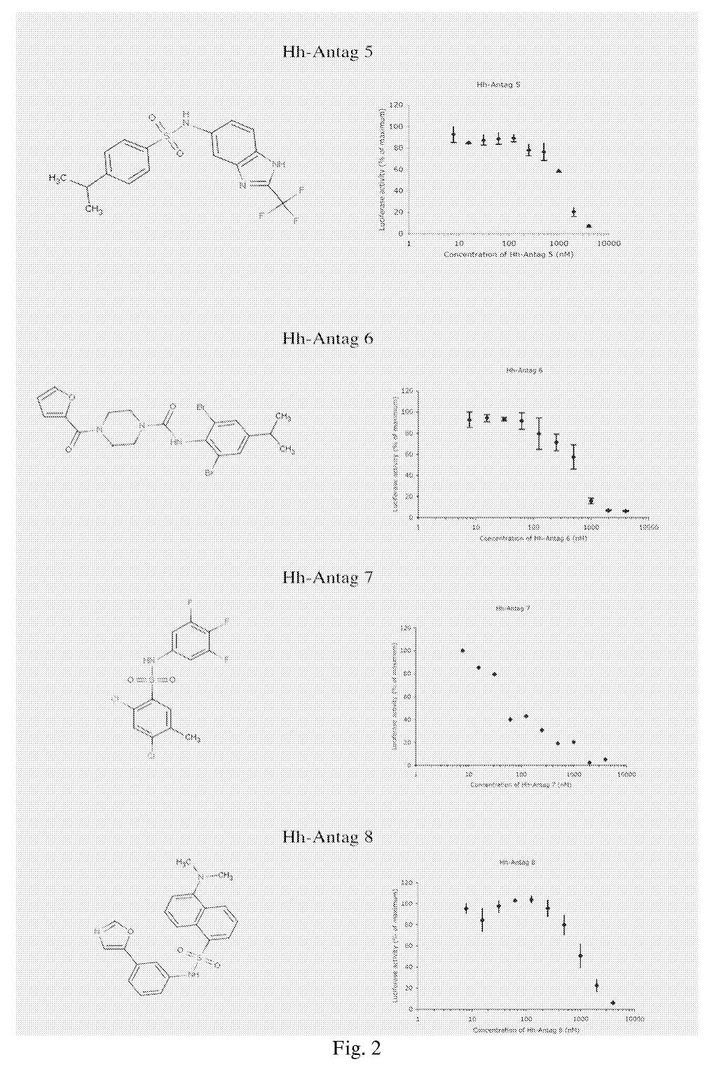

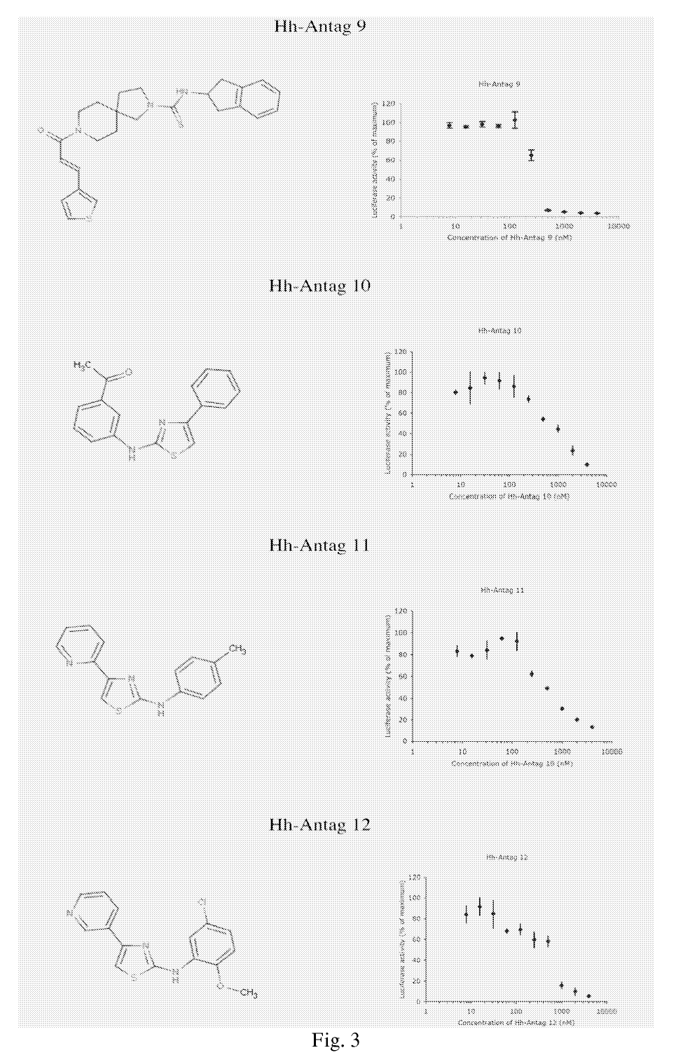

The compounds identified in the 6000 compound assay were tested in secondary assays to measure their individual IC₅₀ values. The graphs of the IC₅₀ data for the 12 compounds identified in the assay are shown in FIGS. 1, 2, and 3. A control experiment was also performed to rule out inhibition of the marker enzyme (Luciferase) in the initial assay and confirm that the compounds identified inhibited the Shh pathway.

### Example 2

### Cell Culture and Animal Studies

The inhibitor (e.g., Shh inhibitory) activity of compounds that are described herein is confirmed using additional cell culture and animal studies. The ability of a compound to inhibit the Hh pathway in vivo is evaluated in model organisms such as zebrafish, mice, rats and chickens. The ability of a compound to inhibit the Hh pathway in vitro is evaluated in cultures of cells derived from fish, avian and mammalian species, including human.

A number of exemplary assays are described in Incardona J P, Gaffield W, Kapur R P, Roelink H., “The teratogenic Veratrum alkaloid cyclopamine inhibits sonic hedgehog signal transduction,” Development. 1998 September; 125(18):3553-62; Chen J K, Taipale J, Young K E, Maiti T, Beachy P A, “Small molecule modulation of Smoothened activity,” Proc Natl Acad Sci USA. 2002 Oct. 29; 99(22):14071-6; and Frank-Kamenetsky M, Zhang X M, Bottega S, Guicherit O, Wichterle H, Dudek H, Bumcrot D, Wang F Y, Jones S, Shulok J, Rubin L L, Porter J A,. “Small-molecule inhibitors of Hedgehog signaling: identification and characterization of Smoothened agonists and antagonists,” J. Biol. 2002 Nov. 6; 1(2): 10; all of which are incorporated by reference for their teachings relating to assays and experimental procedures.

### Example 3

### Cell Culture, Animal, and Human Disease Studies

The efficacy of inhibitory compounds described herein for treatment of cancers is demonstrated using accepted cell lines and animal models, and successful preclinical work is verified in humans. Specific diseases for testing include, but not limited to, the following cases: basal cell carcinomas (BCC) (Ruiz i Altaba et al. 2002; Johnson et al. 1996; Unden et al. 1996), primitive neuroextodermal tumours (PNET), including medulloblastoma (Taylor et al. 2002; Raffel et al. 1997; Peitsch et al. 1997), digestive and gastrointestinal cancers (Berman et al. 2003; Ma et al. 2005; Ma et al. 2006), Colorectal cancer (Qualtrough D et al. 2004); ovarian fibromas and ovarian dermoids (Levanat et al. 2004), oral squamous cell carcinoma (OSCC) (Nishimaki et al. 2004; Michimukai E 2001), small-cell lung cancer (SCLC) (Watkins et al. 2003), prostate cancer (Sanchez et al. 2004), rhabdomyosarcomas (Hahn et al. 2000); human lung squamous carcinomas (HLSC) (Fujita et al. 1997); trichoepitheliomas (TE) (Vorechovsky et al. 1997); Multiple Self-healing Squamous Epitheliomatas (MSSE) (Richards et al. 1997); Rhabdomyosarcomas (RMS) (Hahn et al. 1998); Transitional Cell Carcinomas (TCC) (McGarvey et al. 1998); Dermatofibromas (Leong P M et al. 1999); Trichoblastomas (Aszterbaum M et al., 1999); Odontogenic Keratocyst (OKC) (Barreto et al. 2000); Palmar epidermoid cyst, milia and maxillary cysts (Ogata K et al. 2001); Enchondromatosis (Ollier and Maffucci diseases), including chondrosarcoma (Hopyan S et al. 2002); Mesenchymal hepatic tumors (Koch C A 2002); Human Squamous Cell Carcinoma (SCC) (Koike C et al. 2002); Glioblastoma (Ruppert J M et al. 1988); Meningiomas and Astrocytomas (Salgaller M et al. 1991); Neurofibromatosis, including malignant peripheral nerve sheath tumour (Endo H et al. 2002); Bladder cancer, including urothelial carcinoma (UC) (Aboulkassim T O et al., 2003); pancreatic cancer (Thayer et al. 2003); Ameloblastoma (Kumamoto H et al. 2004); Breast cancer (Kubo M et al. 2004); Pituitary tumors (Vila G et al. 2005); Glioblastoma multiforme, prostate cancer, malignant melanoma and endometrial cancer (Stone, A et al 1999).

Compounds activating the pathway can be used to treat diseases resulting from aberrant inactivation of the Shh pathway in adults as exemplified but not limited in the following: Parkinson's disease (Tsuboi & Shults 2002, Wang et al. 1995 & Flynes ci al. 1995; Bezard 2003).

Compounds activating the pathway can be used to treat diseases resulting from aberrant inactivation of the Shh pathway in development as exemplified but not limited in the following: holoprosencephaly (Roessler et al. 1996 & Ming et al. 2002).

Inhibitors of the hh pathway also may be useful for treating chronic pancreatitis (Kayed et al. 2003); Alzheimer's disease (Reilly et al. 2002); bone regeneration following fracture (Murakami S et al. 2000); neural regeneration (Pepinsky et al. 2002); and spinal cord regeneration (Bambakidis et al. 2004).

As already discussed above, the compounds can be used alone or in combination with other molecules modulating the activity of Shh pathway.

The compounds can be used also as chemicals in biomedical research.

### REFERENCES

The following documents cited herein are incorporated by reference where they are cited.
Aboulkassim T O, LaRue H, Lemieux P, Rousseau F, Fradet Y. Alteration of the PATCHED locus in superficial bladder cancer. Oncogene. 2003 May 15; 22(19):2967-71.
Aszterbaum M, Epstein J, Oro A, Douglas V, LeBoit P E, Scott M P, Epstein E H Jr. Ultraviolet and ionizing radiation enhance the growth of BCCs and trichoblastomas in patched heterozygous knockout mice. Nat. Med. 1999 November; 5(11):1285-91.
Bambakidis N C, Miller R H. Transplantation of oligodendrocyte precursors and sonic hedgehog results in improved function and white matter sparing in the spinal cords of adult rats after contusion. Spine J. 2004 January-February; 4(1):16-26.
Barreto D C, Gomez R S, Bale A E, Boson W L, De Marco L. PTCH gene mutations in odontogenic keratocysts. J Dent Res. 2000 June; 79(6):1418-22.
Berman D M, Karhadkar S S, Maitra A, Montes De Oca R, Gerstenblith M R Briggs K, Parker A R, Shimada Y, Eshleman J R, Watkins D N, Beachy P A. Widespread requirement for Hedgehog ligand stimulation in growth of digestive tract tumours. Nature. 2003 Oct. 23; 425(6960):846-51. 2003 Sep. 14.
Bezard E, Baufreton J, Owens G, Crossman A R, Dudek H, Taupignon A, Brotchie J M. Sonic hedgehog is a neuroinhibitor in the adult subthalamic nucleus. FASEB J. 2003 December; 17(15):2337-8. Epub 2003 Oct 2.
Chen J K, Taipale J, Cooper M K, Beachy P A. Inhibition of Hedgehog signaling by direct binding of cyclopamine to Smoothened. Genes Dev. 2002 Nov. 1; 16(21): 2743-8.
Chen J K, Taipale J, Young K E, Maiti T, Beachy P A. Small molecule modulation of Smoothened activity. Proc Natl Acad Sci USA. 2002 Oct. 29; 99(22):14071-6. Epub 2002 Oct. 21.
Cooper M K, Porter J A, Young K E, Beachy P A. Teratogen-mediated inhibition of target tissue response to Shh signaling. Science. 1998 Jun. 5; 280(5369): 1603-7.
Endo H, Utani A, Shinkai H. Desert hedgehog signalling pathway is involved in the proliferation of a malignant peripheral nerve sheath tumour-derived cell line from neurofibromatosis type 1. Br J Dermatol. 2002 October; 147(4):821-2.
Goodrich L V, Milenkovic L, Higgins K M, Scoff M P. Altered neural cell fates and medulloblastoma in mouse patched mutants. Science. 1997 Aug. 22; 277(5329):l 109-13.
Fujita E, Khoroku Y, Urase K, Tsukahara T, Momoi M Y, Kumagai H, Takemura T, Kuroki T, Momoi T. Involvement of Sonic hedgehog in the cell growth of LK-2 cells, human lung squamous carcinoma cells. Biochem Biophys Res Commun. 1997 Sep. 18; 238(2):658-64.
Hahn H, Wojnowski L, Zimmer A M, Hall J, Miller G, Zimmer A. Rhabdomyosarcomas and radiation hypersensitivity in a mouse model of Gorlin syndrome. Nat. Med. 1998 May; 4(5):619-22.
Hynes M, Porter J A, Chiang C, Chang D, Tessier-Lavigne M, Beachy P A, Rosenthal A. Induction of midbrain dopaminergic neurons by Sonic hedgehog. Neuron. 1995 July; 15(1):35-44.
Hahn H, Wojnowski L, Specht K, Kappler R, Calzada-Wack J, Potter D, Zimmer A, Muller U, Samson E, Quintanilla-Martinez L, Zimmer A. Patched target Igf2 is indispensable for the formation of medulloblastoma and rhabdomyosarcoma J Biol. Chem. 2000 Sep. 15; 275(37):28341-4.
Hopyan S, Gokgoz N, Poon R, Gensure R C, Yu C, Cole W G, Bell R S, Juppner H, Andrulis I L, Wunder J S, Alman B A. A mutant PTH/PTHrP type I receptor in enchondromatosis. Nat. Genet. 2002 March; 30(3):306-10.
Ingham P W, McMahon A P. Hedgehog signaling in animal development: paradigms and principles. Genes Dev. 2001 Dcc 1; 1S(23):3059-87.
Johnson R L, Rothman A L, Xie J, Goodrich L V, Bare J W, Bonifas J M, Quinn A G, Myers R M, Cox D R, Epstein E H Jr, Scott M P. Human homolog of patched, a candidate gene for the basal cell nevus syndrome. Science. 1996 Jun. 14; 272(5268):1668-71.
Kayed H, Kleeff J, Keleg S, Buchler M W, Friess H. Distribution of Indian hedgehog and its receptors patched and smoothened in human chronic pancreatitis. J Endocrinol. 2003 September; 178(3):467-78.
Koch C A, Chrousos G P, Chandra R, Evangelista R, Gilbert J C, Nobuhara K, Zhuang Z, Vortmeyer A O. Two-hit model for tumorigenesis of nevoid basal cell carcinoma (Gorlin) syndrome-associated hepatic mesenchymal tumor. Am J Med. Genet. 2002 Apr. 15; 109(1):74-6.
Koike C, Mizutani T, Ito T, Shimizu Y, Yamamichi N, Kameda T, Michimukai E, Kitamura N, Okamoto T, Iba H. Introduction of wild-type patched gene suppresses the oncogenic potential of human squamous cell carcinoma cell lines including A431. Oncogene. 2002 Apr. 18; 21(17):2670-8.
Kubo M, Nakamura M, Tasaki A, Yamanaka N, Nakashima H, Nomura M, Kuroki S, Katano M. Hedgehog signaling pathway is a new therapeutic target for patients with breast cancer. Cancer Res. 2004 Sep. 1; 64(17):6071-4.
Kumamoto H, Ohki K, Ooya K. Expression of Sonic hedgehog (SHH) signaling molecules in ameloblastomas. J Oral Pathol Med. 2004 March; 33(3): 185-90.
Leong P M, Kauffman C L, Moresi J M, Wu L, Jeronimo C, Sidransky D M, Miller S J. Basal cell carcinoma-like epidermal changes overlying dermatofibromas often reveal loss of heterozygosity in the PTCH gene. J Invest Dermatol. 1999 August; 113(2):279-80
Levanat S, Musani V, Komar A, Oreskovic S. Role of the hedgehog/patched signaling pathway in oncogenesis: a new polymorphism in the PTCH gene in ovarian fibroma. Ann N Y Acad Sci. 2004 December; 1030:134-43.
Ma X, Chen K, Huang S, Zhang X, Adegboyega P A, Evers B M, Zhang H, Xie J. Frequent activation of the hedgehog pathway in advanced gastric adenocarcinomas. Carcinogenesis. 2005 October; 26(10):1698-705. 2005 May 19.
Ma X, Sheng T, Zhang Y, Zhang X, He J, Huang S, Chen K, Sultz J, Adegboyega P A, Zhang H, Xie J. Hedgehog signaling is activated in subsets of esophageal cancers. Int J Cancer. 2006 Jan. 1; 118(1):139-48.
McGarvey T W, Maruta Y, Tomaszewski J E, Linnenbach A J, Malkowicz S B. PTCH gene mutations in invasive transitional cell carcinoma of the bladder. Oncogene. 1998 Sep. 3; 17(9):1167-72.
Meth, M J, Weinberg J M. Cyclopamine: inhibiting hedgehog in the treatment of psoriasis. Cutis. 2006 September; 78(3):158-8.
Michimukai E, Kitamura N, Zhang Y, Wang H, Hiraishi Y, Sumi K, Hayashido Y, Toratani S, Okamoto T. Mutations in the human homologue of the Drosophila segment polarity gene patched in oral squamous cell carcinoma cell lines. In Vitro Cell Dev Biol Anim. 2001 July-August; 37(7):459-64.
Ming J E, Kaupas M E, Roessler E, Brunner H G, Golabi M, Tekin M, Stratton R F, Sujansky E, Bale S J, Muenke M. Mutations in PATCHED-1, the receptor for SONIC HEDGEHOG, are associated with holoprosencephaly. Hum Genet. 2002 April; 11 0(4):297-301.
Murakami S, Noda M. Expression of Indian hedgehog during fracture healing in adult rat femora. Calcif Tissue Int. 2000 April; 66(4):272-6.
Nishimaki H, Kasai K, Kozaki K, Takeo T, Ikeda H, Saga S, Nitta M, Itoh G. A role of activated Sonic hedgehog signaling for the cellular proliferation of oral squamous cell carcinoma cell line. Biochem Biophys Res Commun. 2004 Feb. 6; 314(2):313-20.
Ogata K, Ikeda M, Miyoshi K, Yamamoto Y, Yamamoto T, Osaki T, Michimukai E, Tanaka Y, Sakamoto A, Oakamoto T, Kodama H. Naevoid basal cell carcinoma syndrome with a palmar epidermoid cyst, milia and maxillary cysts. Br J Dermatol. 2001; 145(3):508-9.
Pepinsky R B, Shapiro R I, Wang S, Chakraborty A, Gill A, Lepage D J, Wen D, Rayhorn P, Horan G S, Taylor F R, Garber E A, Galdes A, Engber T M. Long-acting forms of Sonic hedgehog with improved pharmacokinetic and pharmacodynamic properties are efficacious in a nerve injury model. J Pharm Sci. 2002 February; 91(2):371-87.
Pietsch T, Waha A, Koch A, Kraus J, Albrecht S, Tonn J, Sorensen N, Berthold F, Henk B, Schmandt N, Wolf H K, von Deimling A, Wainwright B, Chenevix-Trench G, Wiestler O D, Wicking C. Medulloblastomas of the desmoplastic variant carry mutations of the human homologue of Drosophila patched. Cancer Res. 1997 Jun. 1; 57(11):2085-8.
Pomeroy S L, Tamayo P, Gaasenbeek M, Sturla L M, Angelo M, McLaughlin M E, Kim J Y, Goumnerova L C, Black P M, Lau C, Allen J C, Zagzag D, Olson J M, Curran T, Wetmore C, Biegel J A, Poggio T, Mukherjee 5, Rificin R, Califano A, Stolovitzky G, Louis D N, Mesirov J P, Lander E S, Golub T R. Prediction of central nervous system embryonal tumour outcome based on gene expression. Nature. 2002 Jan. 24; 415(6870):436-42.
Qualtrough D, Buda A, Gaffield W, Williams A C, Paraskeva C. Hedgehog signalling in colorectal tumour cells: induction of apoptosis with cyclopamine treatment. Int J Cancer. 2004 Jul. 20; 110(6):831-7.
Raffel C, Jenkins R B, Frederick L, Hebrink D, Alderete B, Fults D W, James C D. Sporadic medulloblastomas contain PTCH mutations. Cancer Res. 1997 Mar. 1; 57(5):842-5.
Reilly J O, Karavanova I D, Williams K P, Mahanthappa N K, Allendoerfer K L. Cooperative effects of Sonic Hedgehog and NGF on basal forebrain cholinergic neurons. Mol Cell Neurosci. 2002 January; 19(1):88-96.
Richards F M, Goudie D R, Cooper W N, Jene Q, Barroso I, Wicking C, Wainwright B J, Ferguson-Smith M A. Mapping the multiple self-healing squamous epithelioma (MSSE) gene and investigation of xeroderma pigmentosum group A (XPA) and PATCHED (PTCH) as candidate genes. Hum Genet. 1997 December; 101(3):317-22.
Roessler E, Belloni E, Gaudenz K, Vargas F, Scherer S W, Tsui L C, Muenke M. Mutations in the C-terminal domain of Sonic Hedgehog cause holoprosencephaly. Hum Mol. Genet. 1997 October; 6(11): 1847-53.
Ruiz i Altaba A, Sanchez P, Dahmane N. Gli and hedgehog in cancer: tumours, embryos and stem cells. Nat Rev Cancer. 2002 May; 2(5):361-72.
Ruppert J M, Kinzler K W, Wong A J, Bigner S H, Kao F T, Law M L, Seuanez H N, O'Brien S J, Vogelstein B. The GLI-Kruppel family of human genes. Mol Cell Biol. 1988 August; 8(8):3104-13.
Salgaller M, Pearl D, Stephens R. In situ hybridization with single-stranded RNA probes to demonstrate infrequently elevated gli mRNA and no increased ras mRNA levels in meningiomas and astrocytomas. Cancer Lett. 1991 May 24; 57(3):243-53.
Sanchez P, Hernandez A M, Stecca B, Kahler A J, DeGueme A M, Barrett A, Beyna M, Datta M W, Datta S, Ruiz i Altaba A. Inhibition of prostate cancer proliferation by interference with SONIC HEDGEHOG-GLI1 signaling. Proc Natl Acad Sci USA. 2004 Aug. 24; 101(34):12561-6. 2004 Aug. 16.
Stone, A et al., Characterization of the human suppressor of fused, a negative regulator of the zinc-finger transcription factor Gli. J. Cell Sci. 112 (1999), pp. 4437-4448.
Surace E M, Balaggan K S, Tessitore A, Mussolino C, Cotugno G, Bonetti C, Vitale A, Ali R R Auricchio A. Inhibition of ocular neovascularization by hedgehog blockade. Mol. Ther. 2006 March; 13(3):573-9. Epub 2005 Dec. 15.
Taipale J, Beachy P A. The Hedgehog and Wnt signalling pathways in cancer. Nature. 2001 May 17; 411(6835):349-54.
Taylor M D, Liu L, Raffel C, Hui C C, Mainprize T G, Zhang X, Agatep R, Chiappa 5, Gao L, Lowrance A, Hao A, Goldstein A M, Stavrou T, Scherer S W, Dura W T, Wainwright B, Squire J A, Rutka J T, Hogg D. Mutations in SLJFU predispose to medulloblastoma. Nat. Genet. 2002, Jul. 3 1(3):306-10.
Thayer S P, di Magliano M P, Heiser P W, Nielsen C M, Roberts D J, Lauwers G Y, Qi Y P, Gysin S, Fernandez-del Castillo C, Yajnik V, Antoniu B, McMahon M, Warshaw A L, Hebrok M. Hedgehog is an early and late mediator of pancreatic cancer tumorigenesis. Nature. 2003 Oct. 23; 425(6960):851-6.
Tsuboi K, Shults C W. Intrastriatal injection of sonic hedgehog reduces behavioral impairment in a rat model of Parkinson's disease. Exp Neurol. 2002 January; 173(1):95-104.
Unden A B, Holmberg E, Lundh-Rozell B, Stahle-Backdahl M, Zaphiropoulos P G, Toftgard R, Vorechovsky I. Mutations in the human homologue of Drosophila patched (PTCH) in basal cell carcinomas and the Gorlin syndrome: different in vivo mechanisms of PTCH inactivation. Cancer Res. 1996 Oct. 15; 56(20):4562-5.
Vila G, Theodoropoulou M, Stalla J, Tonn J C, Losa M, Renner U, Stalla G K, Paez-Pereda M. Expression and function of Sonic hedgehog pathway components in pituitary adenomas: evidence for a direct role in hormone secretion and cell proliferation. J Clin Endocrinol Metab. 2005.
Vorechovsky I, Unden A B, Sandstedt B, Toftgard R, Stahle-Backdahl M. Trichoepitheliomas contain somatic mutations in the overexpressed PTCH gene: support for a gatekeeper mechanism in skin tumorigenesis. Cancer Res. 1997 Nov. 1; 57(21):4677-81.
Wang M Z, Jin P, Bumcrot D A, Mango V, McMahon A P, Wang E A, Woolf T, Pang K. Induction of dopaminergic neuron phenotype in the midbrain by Sonic hedgehog protein. Nat. Med. 1995 November; 1(j11):1 184-8.
Watkins D N, Berman D M, Burkholder S G, Wang B, Beachy P A, Baylin S B. Hedgehog signalling within airway epithelial progenitors and in small-cell lung cancer. Nature. 2003 Mar. 20; 422(6929):313-7.
Wechsler-Reya R, Scoff M P. The developmental biology of brain tumors. Annu Rev Neurosci. 2001; 24:385-428.

1. A composition comprising (1) a mammalian Hedgehog signaling inhibitor or pharmaceutically acceptable salt, solvate, ester, or prodrug thereof and (2) a pharmaceutically acceptable carrier, diluent, or adjuvant,
wherein the mammalian Hedgehog signaling inhibitor comprises a compound selected from the group consisting of: 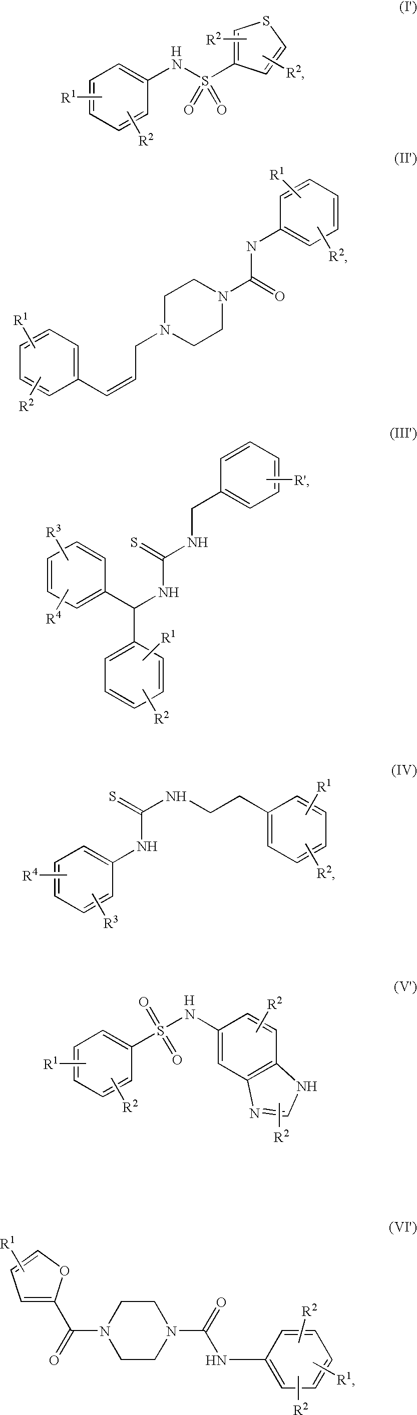 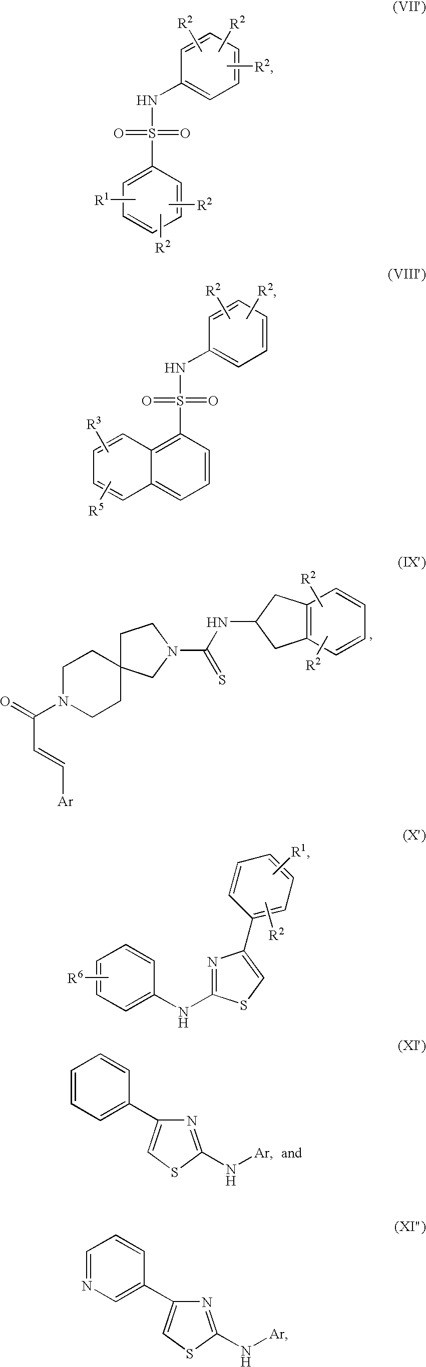
or a pharmaceutically acceptable salt, solvate, ester, or prodrug thereof,
wherein R¹ is hydrogen or unsubstituted or substituted, linear or branched C₁₋₁₀ alkyl;
R² is selected from the group consisting of hydrogen, halogen, unsubstituted or substituted, linear or branched C₁₋₁₀ alkyl, and C₁₋₁₀ alkoxy;
R³ is selected from the group consisting of hydrogen, halogen, and unsubstituted or substituted, linear or branched C₁₋₁₀ alkyl groups;
R⁴ is selected from the group consisting of hydrogen, halogen, unsubstituted or substituted, linear or branched C₁₋₁₀ alkyl, and C₁₋₁₀ alkoxy groups;
R⁵ is selected from the group consisting of hydrogen, halogen, unsubstituted or substituted, linear or branched C₁₋₁₀ alkyl groups, and N(R¹)₂;
R⁶ is selected from the group consisting of hydrogen, halogen, unsubstituted or substituted, linear or branched C₁₋₁₀ alkyl groups, N(R¹⁾₂, and C(O)R¹;
R′ is selected from the group consisting of hydrogen, halogen, unsubstituted or substituted, linear or branched C₁₋₁₀ alkyl, C₁₋₁₀ alkoxy, and SO₂R¹;
R″ is hydrogen, halogen, unsubstituted or substituted linear or branched C₁₋₁₀ alkyl, or unsubstituted or substituted aryl or heteroaryl, and
Ar is unsubstituted or substituted aryl or unsubstituted or substituted heteroaryl.

2. The composition of claim 1, wherein the mammalian Hedgehog signaling inhibitor is selected from the group consisting of: 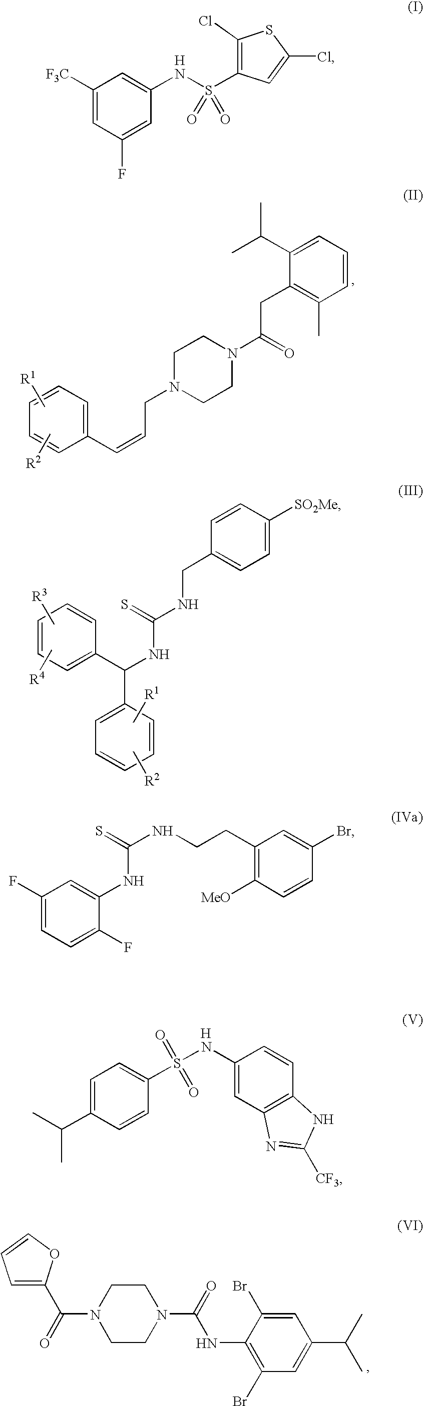 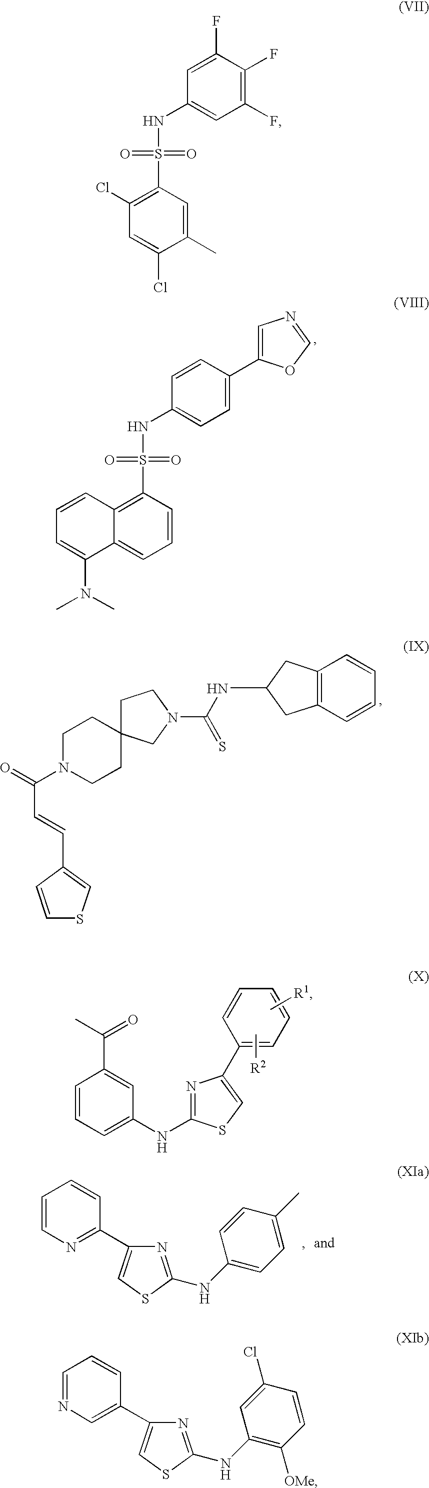
or a pharmaceutically acceptable salt, solvate, ester, or prodrug thereof.

3. The composition of claim 1, wherein R¹ is selected from the group consisting of hydrogen, ethyl, n-propyl, and n-amyl; and R² is selected from the group consisting of hydrogen, chlorine, methyl, and methoxy.

4. The composition of claim 1, wherein R² is selected from the group consisting of hydrogen, ethyl, n-propyl, and n-amyl; and R⁴ is selected from the group consisting of hydrogen, chlorine, methyl, and methoxy.

5. The composition of claim 1, wherein Ar is selected from the group consisting of phenyl, naphthyl, biphenyl, diphenyl ether, diphenylamine, benzophenone, pyrrolyl, pyrrolidinyl, pyridinyl, quinolinyl, indolyl, pyrimidinyl, furyl, thiophenyl, oxazolyl, azolyl, imidazolyl, 1,2,4-triazolyl, tetrazolyl, 2,2′-bipyridinyl, pyridine[3,2,h]quinolinyl, and substituted variants thereof.

6. The composition of claim 1, wherein the mammalian Hedgehog signaling inhibitor comprises formula (XI′) or (XI″).

7. The composition of claim 6, wherein Ar is a phenyl substituted with 1, 2, or 3 substituents selected from the group consisting of halogen, C₁₋₄ alkyl, and C₁₋₄ alkoxy.

8.-18. (canceled)

19. A method of inhibiting a Hedgehog signaling pathway comprising administering to a subject in need thereof a composition according to claim 1 in an amount effective to inhibit the Hedgehog signaling pathway.

20. The method of claim 19 wherein the subject is a mammal.

21. The method of claim 20 wherein the mammal is human.

22. The method of claim 19, wherein the composition comprises about 0.1 to about 20 mg/kg of the mammalian Hedgehog signaling inhibitor.

23.-26. (canceled)

27. The method of claim 19, wherein the subject suffers from a proliferative disorder.

28. (canceled)

29. The method of claim 27, wherein the proliferative disorder is selected from the group consisting of malignant glioma, breast cancer, basal cell carcinoma, medulloblastoma, neuroectodermal tumor, gastrointestinal cancer, ovarian fibroma, ovarian dermoids, oral squamous cell carcinoma (OSCC), small-cell lung cancer (SCLC), prostate cancer, rhabdomyosarcomas, and ependymoma.

30. A method of treating a patient suffering from a cell proliferative disorder comprising selecting the patient in need of treatment for a proliferative disorder and administering to said patient a composition according to claim 1.

31. The method of claim 30, wherein the selecting comprises identifying the proliferative disease by reviewing the patient's medical records, physically examining the patient, performing a diagnostic test, or mixtures thereof.

32. The method of claim 31, wherein the selecting further comprises screening the patient or a biological sample from the patient for aberrant Shh pathway activity, where the selecting comprises choosing the patient with the proliferative disorder and the aberrant Shh pathway activity.

33. The method of claim 31, wherein the biological sample is a biopsy.
